(19) United States
(12) Patent Application Publication
(10) Pub. No.: US 2022/0409560 A1
(43) Pub. Date: Dec. 29, 2022

(54) Pharmaceutical Compositions, Formulations And Methods For The Treatment Of Retinoblastoma

(71) Applicant: Oxular Limited, Oxfordshire (GB)

(72) Inventors: Peter Charlton, Oxfordshire (GB); Afsoon Fallahi, Oxfordshire (GB); Tien Nguyen, Oxfordshire (GB); Ronald Yamamoto, Oxfordshire (GB)

(21) Appl. No.: 17/780,984

(22) PCT Filed: Nov. 27, 2020
(86) PCT No.: PCT/GB20/53062

§ 371 Date: May 29, 2022

(30) Foreign Application Priority Data
Nov. 29, 2019 (GB) 1917487.9

(51) Int. Cl. A61K 31/167; A61K 9/00; A61K 47/24; A61K 47/20; A61K 31/4745; A61P 35/00

(52) U.S. Cl. CPC A61K 31/167; A61K 9/0019; A61K 9/0048; A61K 47/24; A61K 47/20; A61K 31/4745; A61P 35/00

(57) ABSTRACT

The present invention provides a method for the treatment of retinoblastoma comprising administering a composition comprising a therapeutically active agent to a subject in need thereof by injection of the composition into the vitreous cavity, suprachoroidal space, supraciliary space or sub-Tenon's space of the eye adjacent to a retinoblastoma tumour. The invention also provides a composition comprising at least one therapeutically active agent selected from the group consisting of a Bcl-2 inhibitor or a topoisomerase inhibitor for use in the treatment of retinoblastoma, wherein the composition is for administration into the vitreous cavity, suprachoroidal space, sub-Tenon's space, or supraciliary space adjacent to a retinoblastoma tumour in an eye. Also provided is a kit comprising a Bcl-2 inhibitor and a topoisomerase inhibitor for use in the treatment of retinoblastoma, wherein the Bcl-2 inhibitor and the topoisomerase inhibitor are for separate, simultaneous or sequential administration. The invention also provides a kit comprising a composition comprising at least one therapeutically active agent and a cannulation or catheterization device for use in the treatment of retinoblastoma.

The present invention provides compositions, formulations, and methods for the treatment of retinoblastoma.

### BACKGROUND

Retinoblastoma is a cancer that occurs as a result of a genetic mutation of the RB1 gene in the retina resulting in a tumour mass on the retina, with 8,000 new cases diagnosed globally each year leading to approximately 4,000 related deaths. The disease presents in the immature retina resulting in tumours and lesions in children, typically diagnosed at the age of two.

Retinoblastoma may be treated by several different modalities, surgery, chemotherapy and radiotherapy. Surgical treatment may be performed by enucleation for advanced disease, and focal consolidative measures for smaller tumours by laser photocoagulation, thermotherapy and cryotherapy. Radiotherapy treatment may be performed by brachytherapy or external beam radiotherapy.

The most common treatment for retinoblastoma is intravenous chemotherapy, which is associated with serious side effects, the most significant being life-long hearing loss, immune system impairment and mental regression. Children suffer months of acute effects, including severe neutropenia, severe nausea, vomiting, malaise, and hair loss. An alternative is the use of intra-arterial chemotherapy. This procedure involves threading a catheter from the femoral artery in the groin to the ophthalmic branch of the internal carotid artery and delivering chemotherapy directly into the ophthalmic artery and thereby to the tumour. While reducing systemic exposure to chemotherapeutics, this highly invasive procedure has the potential for stroke and ophthalmic artery occlusions. The alternatives are limited, intravitreal injections have poor bioavailability to retinal tumours and require high drug concentrations at retinal toxicity levels to achieve clinical effect. Current treatments report a 17-45% rate of disease recurrence. Treatments tend to be expensive and require specialised facilities and recurrent hospital stays which limits availability to patients.

Methods have been described to improve the treatment of retinoblastoma and ocular tumours by a variety of approaches. U.S. Pat. No. 7,259,180 (WO 2004/016214) describes linking a therapeutic agent to a xanthophyll carotenoid to create a prodrug and administering a therapeutically effective amount of the prodrug to treat retinoblastoma, cystoid macular edema, exudative age-related macular degeneration, diabetic retinopathy, diabetic macular edema, or inflammatory disorders. U.S. Pat. No. 7,432,357 (WO 2005/070967) describes modified antibodies directed against GD2 that have diminished complement fixation relative to antibody-dependent, cell-mediated cytotoxicity that may be used to treat tumours such as neuroblastoma, glioblastoma, melanoma, small-cell lung carcinoma, B-cell lymphoma, renal carcinoma, retinoblastoma, and other cancers of neuroectodermal origin. U.S. Pat. No. 8,837,675 (WO 2008/118198) describes methods for radiation treatment of a target region such as a tumour in an eye to reduce the radiation exposure to the exterior surface of the eye to less than the dose to a target tumour. US/8,470,785 describes the use of nutlin-3 or a nutlin-3 analog to treat retinoblastoma. U.S. Pat. No. 10,117,947 (WO 2015/042325) describes methods and compositions for the diagnosis and/or treatment of tumours, such as ocular tumours, using virus-like particles conjugated to photosensitive molecules. The virus-like particles are administered to the vitreous or intravenously and subsequently the cancerous cells of the tumor are irradiated by an infrared laser. U.S. Pat. No. 10,767,182 (WO 2016/075333) describes selective inhibition of MDM4, e.g., by antisense RNA to treat cancers with high MDM4 protein levels such as melanoma, breast, colon or lung cancers, glioblastoma, retinoblastoma.

The current state of retinoblastoma treatment results in significant rates of recurrence and is a critical concern for patients. More effective treatment approaches are therefore needed to treat retinoblastoma, prevent spread of the cancer and preserve vision with minimal recurrence and long-term side effects. The present invention provides novel active agent compositions, formulations, and administration methods for the treatment of retinoblastoma.

### SUMMARY OF THE INVENTION

The present invention provides pharmaceutical compositions and formulations for the treatment of a retinoblastoma tumour or lesion comprising a compound with inhibitory activity against retinoblastoma. The present invention also provides methods for the treatment of a retinoblastoma tumour or lesion comprising administering a pharmaceutical composition to a subject in need thereof by injection of the composition into the ocular tissues near to or adjacent to the tumour. The administration of the pharmaceutical composition may be placed into the vitreous cavity, in the suprachoroidal space, supraciliary space or the sub-Tenon space of the eye in a region of the cavity or space near the tumour. The administration may be performed with a delivery or injection device utilizing a needle, trocar, cannula, catheter or a combination thereof to perform a minimally invasive, localized ocular administration of the pharmaceutical composition. Administration of the pharmaceutical composition into the ocular tissues near to or adjacent to the tumour therefore includes a site proximal to the retinoblastoma tumour or lesion. Localizing the pharmaceutical composition provides high concentrations of an active agent at the tumour and minimizes systemic exposure to the active agent.

The pharmaceutical compositions are designed to provide a dose of drug or therapeutic active agent required to safely treat the tumour over a course of treatment with the aim of inhibiting tumour growth and reducing tumour size. In some cases, the treatment with the invention may be used adjunctively or in combination with other treatments to eradicate or control the disease with improved efficacy and safety.

The pharmaceutical compositions may comprise an active agent solubilized, dispersed or suspended in a fluid. The pharmaceutical composition of active agent may be prepared with excipients as a high viscosity or semi-solid formulation. Alternatively, the pharmaceutical composition of active agent may be formulated as a solid composition. The pharmaceutical composition of active agent may also be distributed in the composition as particles. The pharmaceutical composition of active agent may also be prepared with excipients as a colloid or as micelles.

The methods of the invention may comprise administration of a composition of a therapeutic active agent with activity against retinoblastoma cells including DNA damaging agents, HIF inhibitors, mitosis inhibitors, DNA synthesis inhibitors, BMI inhibitors, SYK inhibitors, JAK inhibitors, HDAC inhibitors, MEK inhibitors, topoisomerase inhibitors, Bcl-2 inhibitors or a combination thereof. The treatment may comprise the separate, simultaneous, or subsequent administration of one or more active agents to the eye of the subject. The treatment method may also comprise the separate, simultaneous, or subsequent administration of a topoisomerase inhibitor and a Bcl-2 inhibitor to the subject. Optionally, the methods may further comprise the administration of a DNA damaging agent. Suitable methods of treatment may comprise a 14-day cycle or 28-day cycle, optionally repeated as required, for example over a six-month period.

### DETAILED DESCRIPTION OF THE INVENTION

The invention comprises a composition or formulation comprising a therapeutically active agent for use in the treatment of retinoblastoma by delivery locally to an eye, wherein the administration of the composition or formulation is to a site near to or adjacent to a retinoblastoma tumour or lesion. The invention comprises a composition comprising a topoisomerase inhibitor for use in the treatment of retinoblastoma by administration locally to an eye, wherein the administration of the composition is to a site adjacent to a retinoblastoma tumour or lesion. The invention comprises a composition comprising an HDAC inhibitor for use in the treatment of retinoblastoma by administration locally to an eye, wherein the administration of the composition is to a site adjacent to a retinoblastoma tumour or lesion. The invention comprises a composition comprising a Bcl-2 inhibitor for use in the treatment of retinoblastoma by administration locally to an eye, wherein the administration of the composition is to a site adjacent to a retinoblastoma tumour or lesion. The invention comprises a composition comprising a combination of at least two of a Bcl-2 inhibitor, HDAC inhibitor, and topoisomerase inhibitor for use in the treatment of retinoblastoma by administration locally to an eye, wherein the administration of the composition is to a site near to or adjacent to a retinoblastoma tumour or lesion. The composition or formulation may be administered into the vitreous cavity, suprachoroidal space, sub-Tenon's space, or supraciliary space near to or adjacent to a retinoblastoma tumour in an eye.

Suitably a delivery device may administer a composition of the active agent in a minimally invasive method locally near to or adjacent to the retinoblastoma tumour to provide a high local concentration of the active agent to the tumour and minimise exposure to other ocular tissues and systemically related to effects that may limit treatment. The delivery device may be an injection device utilizing a needle, trocar, cannula, catheter or a combination thereof to perform the minimally invasive localized ocular administration of the active agent. The composition may be delivered to the vitreous cavity, the sub-Tenon's space or the suprachoroidal space in a region near or adjacent the tumour.

The local administration may be performed by injection, infusion, or delivery of an implant containing the therapeutically active agent. The local administration may be administered with a variety of devices including a needle, cannula or catheter. The sites for placement of the therapeutically active agent near or adjacent the tumour includes the vitreous cavity, the suprachoroidal space and the sub-Tenon's space. The ocular drug delivery device used should therefore suitably be designed to precisely deliver drugs or therapeutically active agents near to or adjacent to the specific site of the cancer in the eye. Using such a device permits administration in a less invasive manner than existing treatments, causes significantly less physical trauma to the patient, and mitigates the potential to spread the cancer in the patient's body. Such uses further include the use of a one or more of the aforementioned active agents in the manufacture of a medicament for the treatment of retinoblastoma by administration with a needle, cannula or catheter into a space or region near to or adjacent to a retinoblastoma tumour or lesion.

In particular, the suprachoroidal space overlies the retina and allows the manoeuvring of a flexible cannula or catheter in the space to reach a position overlying a retinoblastoma tumour or lesion. The cannula or catheter may subsequently deliver a therapeutic composition in the space near or adjacent a target tumour or lesion. The distal end of the cannula or catheter may be introduced into the suprachoroidal space or the adjacent supraciliary space and advanced and positioned in the suprachoroidal space. A flexible cannula or catheter may be placed in the suprachoroidal space or supraciliary space from an anterior region such as the pars plana and then advanced into the suprachoroidal space to position the distal tip proximal to the target tumour to prevent possible administration device contact with the tumour and inadvertent spread of the tumour cells. The described use of a cannula or catheter in the suprachoroidal space enables administration of a therapeutic compound near to or adjacent to a retinoblastoma tumour without the risk of tumour spread or tumour extension associated with tumour contact with a device for example from intra tumour injection. Administration of the therapeutic agent into the space near or adjacent to the tumour therefore includes a site proximal to the retinoblastoma tumour or lesion.

A composition is provided for the treatment of retinoblastoma by administration into the suprachoroidal space comprising a Bcl-2 inhibitor. A combined composition or preparation is also provided for the treatment of retinoblastoma by administration into the suprachoroidal space comprising a Bcl-2 inhibitor and a topoisomerase inhibitor or a Bcl-2 inhibitor and an HDAC inhibitor for separate, simultaneous, or sequential administration.

Examples of Bcl-2 inhibitors include but are not limited to TW-37, venetoclax, navitoclax, ABT-737, sabutoclax, obatoclax, ABT-263, oblimersen, AT101, SS5746, APG-1252, APG-2575, S55746 and UBX1967/1325.

Examples of topoisomerase inhibitors include, but are not limited to topotecan, irinotecan, doxorubicin, irinotecan, daunorubicin, SN-38, voreloxin, belotecan and semisynthetic derivatives of podophyllotoxin (etoposide).

Examples of HDAC inhibitors include, but are not limited to vorinostat, belinostat, panobinostat, romidepsin, entinostat, mocetinostat, CUDC-101, tacedinaline or nicotinamide.

Examples of anti-cancer agents include, but are not limited to, 2-methoxystradiol.

Examples of DNA-damaging agents include, but are not limited to, altretamine, bendamustine, busulfan, carboplatin, carmustine, chlorambucil, cisplatin, cyclophosphamide, dacarbazine, dactinomycin, ilfosfamide, lomustine, mechlorethamine, melphalan, oxaliplatin, procarbazine, streptozocin, temozolomide, thiotepa and trabectedin.

The invention also provides a kit comprising a Bcl-2 inhibitor, a HDAC inhibitor and a topoisomerase inhibitor for use in the treatment of retinoblastoma, wherein the Bcl-2 inhibitor, a HDAC inhibitor or the topoisomerase inhibitor are for separate, simultaneous or sequential administration.

The invention also provides a kit comprising a composition comprising at least one therapeutically active agent and a cannulation or catheterization device for use in the treatment of retinoblastoma in an eye, wherein the at least one therapeutically active agent is selected from the group consisting of a Bcl-2 inhibitor, a HDAC inhibitor or a topoisomerase inhibitor and wherein the cannulation or catheterization device is for the suprachoroidal space or supraciliary space, optionally a pharmaceutically acceptable diluent may be present also in the kit.

In one embodiment, the invention provides a Bcl-2 inhibitor, a HDAC inhibitor or a topoisomerase inhibitor which inhibits cell growth, invasion and angiogenesis or promotes apoptosis in retinoblastoma cells.

In one embodiment, the treatment is for a 14 to 28 day cycle of therapy via local ocular administration, with repeated cycles over a six-month period.

The therapeutic active agent may comprise a sustained-release formulation of one or more of the therapeutically active agents to provide active agent release over a period of time. A sustained release formulation may be tailored to provide a therapeutically effective amount of the active agent over a period of time to tailor a treatment interval or cycle.

Suitable active agent compounds for use in the treatment of retinoblastoma with significantly reduced toxicity to normal cells to provide a therapeutic range have been identified in the course of the work described herein. Two promising compounds advanced to an in-vivo retinoblastoma tumour rabbit model.

TW-37—a potent small-molecule inhibitor that attenuates Bcl-2 activation and inhibits multiple Bcl-2 family members. Bcl-2 is anti-apoptotic and proangiogenic protein and inhibition by TW-37 helps induce cancer cell apoptosis

Topotecan—a semisynthetic derivative of the cytotoxic alkaloid, camptothecin. Topotecan inhibits topoisomerase-1, an enzyme involved in DNA replication. Topotecan intercalates between DNA bases in the topoisomerase-I cleavage complex resulting in difficult to repair double strand breaks.

The Bcl-2 inhibitor class of compounds have properties that would benefit from local administration to increase tumour levels of the compound while reducing systemic exposure. This compound class has also been studied in combination with a DNA damaging agent (cisplatin) or a topoisomerase inhibitor to create synergistic or additive cancer treatment effects. Other Bcl-2 inhibitors (venetoclax, navitoclax, ABT-737, sabutoclax, obatoclax, ABT-263, oblimersen, AT101, SS5746, APG-1252, APG-2575, S55746 and UBX1967/1325) may also demonstrate treatment effects similar to TW-37.

The invention provides (i) delivery of a drug product to the retina, the site of retinoblastoma, through minimally invasive delivery; (ii) clinical management of retinoblastoma; (iii) reduction in the number of treatments and related side effects; (iii) reduction in the rate of disease recurrence; and (iv) preservation of sight and avoiding eye enucleation.

Pharmaceutical compositions for use in accordance with any aspect of the invention may comprise the drug composition, excipients and a pharmaceutically acceptable diluent are provided. The pharmaceutically acceptable diluent may comprise salt to provide physiologically acceptable osmolality and pH to the drug composition prepared with the diluent. The pharmaceutically acceptable diluent may contain a reconstitution aid to promote rapid reconstitution of the drug composition in dry form. The pharmaceutical composition for use may be provided in a unit dosage form.

### Formulations of Therapeutic Active Agents

The active agent may be solubilized, dispersed or suspended in a fluid. The active agent may be prepared with excipients as a high viscosity or semi-solid formulation. Alternatively, the active agent may be formulated as a solid composition. The active agent may also be distributed in the composition as particles. The active agent may also be prepared with excipients as a colloid or as micelles.

The formulation may comprise a liquid, solid, semi-solid, colloidal, or micellular active agent composition comprising a therapeutically active agent and excipients as defined herein such that the composition is designed for administration through a small gauge needle, cannula or catheter, for example, placed into the vitreous cavity, suprachoroidal space, sub-Tenon's space of an eye. In the present application the terms “active agent”, “drug”, “therapeutic agent”, “therapeutically active agent”, and “therapeutic material” are used interchangeably. In the context of the present application, a semi-solid composition refers to a material that does not flow without pressure and remains localized to a location in the eye immediately after delivery.

As described herein, in one embodiment, a semi-solid material for injection may comprise active agent particles in a semi-solid excipient or mixture of excipients. In one embodiment, the semi-solid material may comprise solubilized active agent in a semi-solid excipient or mixture of excipients. In particular, the semi-solid composition comprises an active agent; the semi-solid composition flows under injection pressure; the semi-solid composition remains localized at the site of administration near to or adjacent to the retinoblastoma tumour during and immediately after administration; and the semi-solid composition undergoes dissolution over time.

In one embodiment, the active agent or drug is combined with a biodegradable polymer to form active agent containing particles. The biodegradable polymer may be selected from the group consisting of polyhydroxybutyrate, polydioxanone, polyorthoester, polycaprolactone, polycaprolactone copolymers, polycaprolactone-polyethylene glycol copolymers, polylactic acid, polyglycolic acid, polylactic-glycolic acid copolymer and/or polylactic-glycolic acid-ethylene oxide copolymer.

In another embodiment the active agent is present in an amount from 0.5 wt % to 70.0 wt % of the biodegradable polymer and active agent composition, suitably, 10.0 wt % to 60.0 wt %, 15.0 wt % to 50.0 wt %, preferably 20.0 wt % to 40.0 wt %. Suitable active agents are discussed herein above.

In a further embodiment the active agent composition may comprise a salt. The salt may be selected from the group consisting of sodium, potassium, calcium and magnesium salts including phosphates, chloride, carbonates, acetates, citrates, gluconates, carbonates, tartrates and combinations thereof. The salts or combination of salts may be formulated to provide physiological acceptable pH and osmolality. The combination of salts may also be phosphate buffered saline.

In one embodiment, the formulation of the therapeutic active agent is formed into a semi-solid composition that flows upon application of injection pressure but once administered into tissue, forms a semi-solid material at the location of delivery to localize the active agent near the target treatment site. The ability to administer the composition through a small gauge needle, cannula or catheter is aided by the use of excipients that provide viscoelastic properties to promote flow during injection. Suitable viscoelastic excipients include high molecular weight polyethylene glycol, polyethylene oxide, high molecular weight polyvinylpyrrolidone, and biological polymers such as polymeric lipids, hyaluronic acid and chondroitin sulphate. Viscoelastic excipients in the concentration range of 0.3 wt % to 50 wt %, 1 wt % to 40 wt %, 5 wt % to 30 wt %, 10 wt % to 20 wt % percent depending on polymer selection and molecular weight provide injectable compositions. In one embodiment, the composition is formulated with one or more therapeutic active agents and an excipient mixture comprising a viscoelastic excipient and a physiological buffer. In one embodiment the composition comprises an excipient that undergoes dissolution, biodegradation or bioerosion in the vitreous cavity, suprachoroidal space, or sub-Tenon's space after injection.

In one embodiment, a solid or semi-solid composition is formed in a mould or extruded and allowed to dry to form a solid of desired dimensions for administration. Ideal for administration of the formed solid or semi-solid composition is an elongated shape with an outer diameter sized to fit within the lumen of a small diameter cannula or needle, 20 gauge or smaller, corresponding to 0.60 mm (0.02 inches) diameter or smaller. In one embodiment, the formed solid or semi-solid composition has an outer diameter sized to fit within the lumen of a 25 gauge or smaller cannula or needle, corresponding to a 0.26 mm (0.01 inches) diameter or smaller. In one embodiment, the formed solid or semi-solid composition has an outer diameter sized to fit within the lumen of a 27 gauge or smaller cannula or needle, corresponding to a 0.20 mm (0.008 inches) diameter or smaller.

In one embodiment, the active agent composition is prepared as a formulation that produces colloidal or micelle structures that contain or encapsulate the active agent. The active agent may also be associated with the micelles in the outer layer or surface of the micelles. In the present application the terms of active agent encapsulated, contained or associated with the micelles represent the partitioning of the active agent to the micelle structure. The encapsulation or association of the active agent in the micelle provides sustained release of the active agent to extend the therapeutic effect after treatment with the formulation. Encapsulation or association of the active agent in the micelle provides protection of the active agent such as from degradation. The encapsulation may be performed by solubilizing the active agent in an organic solvent or mixture of solvents to form an organic solution. An amphiphilic compound that may form a micellular structure and act as a micelle forming excipient is solubilized in an aqueous solvent to form a second solution. Combining the two solutions in appropriated amounts and mixing of the two solutions results in association of the active agent with the micelle forming excipient resulting in the active agent contained or associated with the micelles suspended in the aqueous solvent. The partitioning of the active agent to the micelles by association or encapsulation results the in the substantial portion of the active agent within and/or associated with the micelles such that the active agent in the aqueous phase is below the solubility limit of the active agent to prevent active agent crystal formation in the aqueous phase. The mixing may be performed by shaking a container containing the composition, vortex mixing, high shear mixing, or sonication to form the micelles. In some formulations, the micelles may form with relatively low shear mixing, or self-assemble. In some formulations, higher energy such as high shear mixing, or sonication is required for micelle formation. The size and concentration of the micelles are controlled by the composition of the formulation including the concentration of the constituents, the partitioning and solubility properties of the amphiphilic excipient, the concentrations of the amphiphilic excipient, the concentration of the active agent, ionic strength and pH of the aqueous solution and the mixing conditions.

The micellular formulation comprises the active agent, the micelle forming excipient, the solvent or mixture of organic solvents for the active agent, and the aqueous solution. The composition may be prepared to provide a final sterilised product. One or more active agents are solubilized in an organic solvent and filter sterilised into a sterile container. One or more micellular excipients are solubilized in the aqueous solvent and filter sterilised. A volume of the sterilised active agent solution is mixed with the sterilised micellular excipient aqueous solution in the appropriate proportions to create an environment for partitioning of the active agent and micelle forming excipient into a micelle discontinuous phase suspended in the aqueous solution continuous phase. The micelles may be formed by mixing the container, a mixing mechanism within the container or by sonication of the composition. The final micellular composition may be subsequently aseptically filled into sterile vials. Alternatively, the active agent and micelle forming excipients may be sterile filtered and dispensed into sterile vials and the appropriate amount of the aqueous solution sterile filtered and added to the vials. The micelles may be formed by mixing of the vials, sonication of the vials or by agitation to promote micelle assembly just prior to use.

Due to the inherent physical instability of micellular formulations, the appropriate excipients and stoichiometry with the active agent is necessary for suitable stability of the micelles to allow for manufacturing, transport and storage. Micelle forming amphiphilic compounds comprising polymers or conjugated polymers may provide enhanced physical stability and sustained release properties when the formulation parameters are balanced with respect to concentrations and stoichiometry of the active agent and the micelle forming excipients. Physical stability is required for the administration of micellular formulations through small gauge needles, cannulas and catheters which may disrupt the micelles by fluid shear through a small lumen. Suitable polymers include polyethylene glycol copolymers and polypropylene glycol copolymers. Suitable conjugated polymers include polyethylene glycol conjugated lipids, polyethylene glycol conjugated phospholipids and polyethylene glycol conjugated sterols.

In one embodiment, an active agent is dissolved in one or more organic solvents to produce a first solution. Suitable solvents include DMSO, dichloromethane and ethyl acetate. A second solution is prepared by dissolving the amphiphilic polymer in an aqueous solvent. Suitable solvents include water, aqueous buffers and aqueous dispersions of hydrophilic polymers. A volume of the organic solvent solution is combined with a volume of the aqueous solution. Mixing of the two solvents produces a micelle formulation with the active agent encapsulated in or associated with the micelles as a discontinuous phase suspended in an aqueous continuous phase. After formation of the micelles containing the active agent, the organic solvents may optionally be removed or reduced in concentration. Micelles with high physical stability may be dried such as by lyophilization or the aqueous continuous phase exchanged with another aqueous solution to slowly remove the organic solvents.

In one embodiment, a Bcl-2 inhibitor, TW-37, is prepared as a micelle formulation. TW-37 is prepared in an organic solvent to solubilize the active agent. Suitable solvents include DMSO, dichloromethane and ethyl acetate. The concentration of the TW-37 in the solvent is prepared for a solution concentration in the range of 3 to 100 mM. A second solution is prepared with a polyethylene glycol (PEG) conjugated lipid in an aqueous solvent. Suitable PEG conjugated lipids include conjugated 1,2-dimyristoyl-sn-glycero-3-phosphoethanolamine (DMPE), conjugated 1,2-Dipalmitoyl-sn-glycero-3-phosphorylethanolamine (DPPE), conjugated 1,2-Distearoyl-sn-glycero-3-phosphorylethanolamine (DSPE), conjugated 1,2-dioleoyl-sn-glycero-3-phosphoethanolamine (DOPE). The concentration of the PEG conjugated lipid in the aqueous solution is typically in the range of 5 mM to 100 mM, 20 mM to 80 mM, 30 mM to 60 mM, 40 mM to 50 mM. The PEG in the conjugated lipid may be of varying chain length to tailor the amphiphilic properties of the conjugated lipid, with greater chain length creating interaction with the aqueous continuous phase of the formulation. The PEG chain length may vary from a molecular weight of 100 to 5000, 200 to 4000, 550 to 3000, 1000 to 2000 Daltons. A volume of the TW-37 containing solution is mixed with a volume of the PEG conjugated lipid containing aqueous solution in the appropriate proportions to create an environment for partitioning of the active agent and micelle forming excipient into a micelle discontinuous phase suspended in the aqueous solution continuous phase. In general, stable formulations are observed with molar stoichiometries of approximately 1:1 PEG conjugated lipid to TW-37, or with the PEG-lipid slightly in excess of 1:1 stoichiometry. Hence the micelle formulation may have stoichiometry of PEG-lipid to TW-37 of 3:1 to 1:3, 2:1 to 1:2 or 3:2 to 2:3. In one embodiment, the PEG-lipid comprises PEG conjugated phospholipids including 1,2-distearoyl-sn-glycero-3-phosphoethanolamine-N-[methoxy(polyethylene glycol)-550] (18:0 PEG550 PE), 1,2-distearoyl-sn-glycero-3-phosphoethanolamine-N-[methoxy(polyethylene glycol)-1000] (18:0 PEG1000 PE), or 1,2-dimyristoyl-sn-glycero-3-phosphoethanolamine-N-[methoxy(polyethylene glycol)-1000] (14:0 PEG1000 PE).

Physical stability of the micellular formulation may be assessed by microscopy to determine the presence of intact, spherical micelles. Loss of physical stability is characterized by the formation of non-spherical particles, aggregates of the micelles, or escape of the active agent from the micelles to form crystals in the aqueous phase. Chemical stability of the micellular formulation is assessed by conventional means such as chemical assay by HPLC, LCMS, NMR or spectroscopy. The stability of the formulation under storage conditions prior to administration is necessary to transport the formulation to provide patient treatment. The stability of the formulation in physiologic conditions is necessary to provide adequate time for the active agent to penetrate into the target tumour cells to provide a therapeutic effect.

The physical stability of the micelle formulations was found to be dependent on the selection of the micelle forming excipient, the concentration of the excipient and the active agent, and especially the ratio of the active agent to the excipient. Final formulation concentrations of the active agent in the range of 3.75 mM to 15 mM, 5 mM to 10 mM, 7 mM to 8 mM and the excipient in the rage of 5 to 15 mM, 8 mM to 12 mM was found to promote stability of the micelle formulations. Typically, the active agent soluble in organic solvent of the active agent containing solution has poor solubility in water. The loss of physical stability of the micelles results in escape of the active agent from the micelles and the formation of crystals in the aqueous phase of the formulation.

Chemical stability of the micelle formulation is dependent on the properties of the active agent. The association of the active agent with the micelles protects the active agent from hydrolytic degradation. Stabilizing agents to limit oxidative degradation may be added to the formulation to provide protection of the active agent in the micelles. Suitable antioxidants include alpha tocopherol and alpha tocopherol derivatives, butylated hydroxy anisole, and butylated hydroxyl toluene.

In one embodiment, the composition may comprise a Bcl-2 inhibitor, an excipient comprising an amphiphilic polymer, and an aqueous solution, wherein the Bcl-2 inhibitor is associated with the excipient in the form of micelles suspended in the aqueous solution. The amphiphilic polymer may comprise a polyethylene glycol conjugated lipid. The polyethylene glycol conjugated lipid may be selected from the group consisting of polyethylene glycol conjugated 1,2-dimyristoyl-sn-glycero-3-phosphoethanolamine (DMPE), conjugated 1,2-Dipalmitoyl-sn-glycero-3-phosphorylethanolamine (DPPE), conjugated 1,2-Distearoyl-sn-glycero-3-phosphorylethanolamine (DSPE) or conjugated 1,2-dioleoyl-sn-glycero-3-phosphoethanolamine (DOPE). The polyethylene glycol in the polyethylene glycol conjugated lipid may have a molecular weight range of 100 to 5000, 200 to 4000, 550 to 3000, 1000 to 2000 Daltons. The Bcl-2 inhibitor may be TW-37. The concentration of the Bcl-2 inhibitor may in the range of 1.5 μM to 50 μM, 5 μM to 40 μM, 10 μM to 30 μM, 15 μM to 20 μM. The concentration of the conjugated lipid is in the range of 2.5 μM to 50 μM, 5 mM to 80 mM, 10 mM to 60 mM. 20 mM to 40 mM. The molar ratio of the Bcl-2 inhibitor to amphiphilic polymer is in the range of 1:3 to 3:1, 1:2 to 2:1 or 2:3 to 3:2. The composition may further comprise a topoisomerase inhibitor. In one embodiment, the conjugated lipid in the polyethylene glycol conjugated lipid is 1,2-dimyristoyl-sn-glycero-3-phosphoethanolamine-N-[methoxy(polyethylene glycol)-1000].

To minimize the frequency of administration to a patient, in some embodiments, the composition may be configured to provide slow release of the drug. The colloidal or micelle structures may be suspended in a viscoelastic excipient to aid flow properties in delivery through a small gauge needle, cannula or catheter. The sizing of the particles and concentration in a semi-solid or viscous excipient enables injection of a small volume through a small gauge needle, cannula or catheter.

In one embodiment, the active agent composition is dried, such as by lyophilisation, spray drying or air drying, to aid shelf life stability and is rehydrated prior to administration. The composition may have excipients to aid reconstitution such as salts, sugars, water soluble polymers and surfactants. For dried formulations, the use of bulking agent such as sucrose, mannitol, glycine, povidone, or dextran, aids the production of a loose dried product with large channels or pores to enhance reconstitution speed. Prior to drying, the bulking agent may be in the concentration range of 1.0 wt % to 20.0 wt %, 1.0 wt % to 10.0 wt % in the excipient mixture. The final dried composition may have a bulking agent in the range of 5 wt % to 50 wt %, 10 wt % to 40 wt %, 20 wt % to 30 wt %. Excipients to increase reconstitution of the dried composition to act as reconstitution aids, such as surfactants, salts, sugars or trehalose may be added prior to drying. The final dried composition may have a reconstitution aid in the range of, 0.1 wt % to 45.0 wt %, 0.1 wt % to 20.0 wt %, 1.0 wt % to 15.0 wt % or 2.0 wt % to 10.0 wt %. The composition may be reconstituted with water or a physiological buffer immediately prior to use. In one embodiment the composition may additionally contain an excipient to speed reconstitution such as trehalose. The combination of the components must be carefully balanced to provide the physical stability to dry the composition without precipitation, aggregation or degradation to subsequently provide rapid rehydration and flow properties for administration through a small lumen. In one embodiment, the composition is formulated to also provide physiologically compatible osmolality, generally in the range of 250 to 450 mOsM, and pH, generally in the range of 7 to 8.

In one embodiment the active agent composition may suitably be present in a substantially dry form and can be considered to be free from water. The active agent composition may be dried using any generally convenient process including lyophilisation and spray drying. The active agent composition may be considered as anhydrous after drying but it is not excluded that a small amount of residual moisture may be present.

The is also provided a method for preparing the composition of the invention comprising: mixing the Bcl-2 inhibitor with one or more organic solvents to dissolve the Bcl-2 inhibitor; filtering sterilely the mixture; adding the organic solvent mixture to a volume of sterile filtered aqueous solution containing the amphiphilic polymer excipient; and mixing the sterile formulated composition to produce Bcl-2 inhibitor containing micelles in an aqueous solution. The Bcl-2 inhibitor may be TW-37. The amphiphilic polymer excipient may be 1,2-dimyristoyl-sn-glycero-3-phosphoethanolamine-N-[methoxy(polyethylene glycol)-1000]. The organic solvent may be DMSO.

### Devices for Local Ocular Administration of Therapeutic Active Agents

In the methods of the present invention, the formulation of the therapeutically active agent is administered by injection to the vitreous cavity, suprachoroidal space or sub-Tenon's space of the eye by a delivery device. Administration of the therapeutically active agent to the suprachoroidal space with a cannula or catheter has particular advantages of positioning the distal tip of the cannula or catheter in the space near the target tumour to direct the volume of active agent adjacent to the target tumour. An example of a suitable device for use in accordance with the present invention is described in WO 2019/053465. Such devices permit delivery of the formulation of the therapeutically active agents via a cannula or catheter in the device and are briefly described as follows.

Placement of a cannula or catheter into the suprachoroidal space or supraciliary space of an eye provides a means to enter the eye with the device in a region distant from the tumour, advance the cannula or catheter to a position in the suprachoroidal space near or adjacent the tumour to be treated and deliver an active agent containing composition. The cannulation or catheterization device allows an active agent containing composition to be administered and directed toward a specific target region near or adjacent the tumour from an anterior tissue access site such as the pars plana. Flexible cannulas and catheters may be introduced in a tissue space such as the suprachoroidal space, sub-Tenon's space, supraciliary space or vitreous cavity and advanced to the desired position adjacent to the target tumour. In the suprachoroidal space, sub-Tenon's space or supraciliary space, the cannula or catheter may be designed and fabricated to conduct light to thereby direct illumination from the distal tip of the device to aid ab-externo and/or ab-interno visualization of the tip of the device in relation to the location of the target tumour. The illumination from the cannula or catheter enables the position of the distal end of the cannula or catheter to be adjusted to a location near the target tumour without contacting the tumour. Illumination of the entire length of the cannula or catheter to include the shaft as well as the distal tip provides confirmation of the direction that the volume of therapeutic agent will be delivered from the device to insure optimal location of the therapeutic agent in relation to the tumour. If the cannula or catheter is curved away from the tumour although the distal tip is near or adjacent the tumour, the volume of administered active agent will be directed away from the tumour. The illumination of the entire length of the cannula or catheter allows both the position of the tip and shaft of the cannula or catheter to be adjusted to direct the active agent formulation toward the target tumour location.

The small gauge size of the cannula or catheter used to administer the active agent containing composition is designed for minimally invasive access to the suprachoroidal space or supraciliary space. The outer diameter of the small gauge cannula or catheter is preferably 25 gauge or smaller (0.51 mm), 27 gauge or smaller (0.41 mm), 30 gauge or smaller (0.30 mm) with inner diameters of approximately 0.35 mm, 0.26 mm, 0.16 mm respectively.

The described embodiments of the cannulation or catheterization device may be used in combination to cannulate or catheterize a tissue space and administer a fluid, semi-solid or solid, including a micellular or colloidal formulation. In one embodiment the configuration of the distal portion of the cannulation or catheterization device comprises a distal element which functions as a tissue interface and distal seal on the distal end of the needle. The cannula or catheter and reservoir for the delivery material may be configured for administration of a fluid, semi-solid, solid or implant from the cannula or catheter. In some embodiments, the lumen of the cannula or catheter may also act as the reservoir or a portion of the reservoir of the active agent formulation.

For controlled administration of the composition containing the therapeutic active agent, the volume delivery from the device must have high accuracy and precision. The injection volumes for localized treatment from a composition delivered adjacent to a tumour range from 10 to 100 microliters, 20 to 90 microlitres, 40 to 70 microlitres, 50 to 60 microlitres depending on the size of the tumour or collection of tumours to be treated. The injection rate, dead space, flow path and mechanical tolerances of the device are designed for a precision of delivery of in the range of at least 20%, at least 15%, at least 10% or at least 5%. The design of flow path and parameters such as injection rate may be tailored to the flow properties such as viscosity and viscoelasticity of the composition for administration. The flow path may be tailored also for shear sensitive active agent formulations such as a suspension of micelles containing the active agent.

Preferred features of the second and subsequent aspects of the invention are as for the first aspect mutatis mutandis.

The invention will now be further described by way of reference to the following Examples and Drawings which are present for the purposes of illustration only.

In the Examples, reference is made to the following Figures in which:

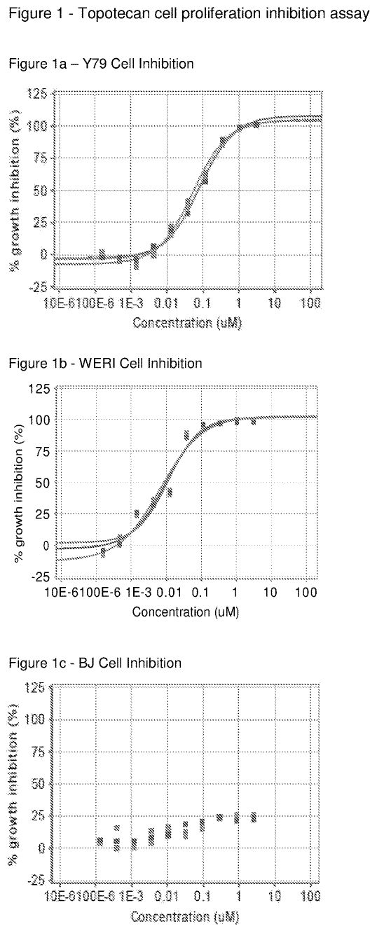

FIG. 1 shows the results of retinoblastoma cell proliferation inhibition assays with a topoisomerase inhibitor (topotecan). FIG. 1a shows results for Y79 cell line. FIG. 1b shows results for WERI cell line. FIG. 1c shows results for BJ cell line.

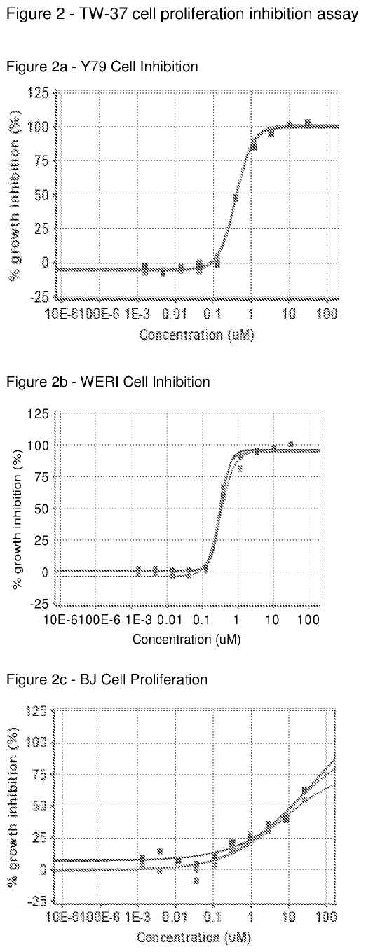

FIG. 2 shows the results of retinoblastoma cell proliferation inhibition assays with a Bcl-2 inhibitor (TW-37). FIG. 2a shows results for Y79 cell line. FIG. 2b shows results for WERI cell line. FIG. 2c shows results for BJ cell line.

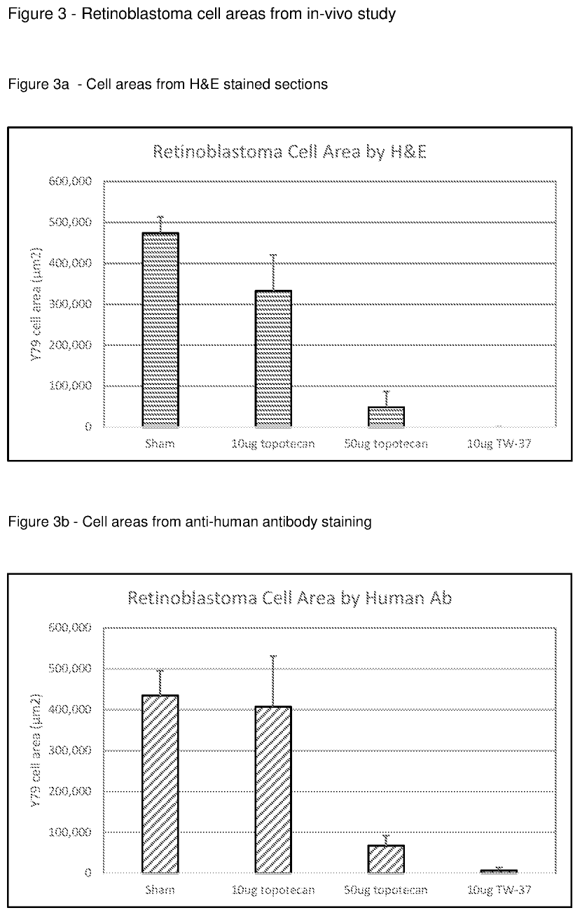

FIG. 3 shows the retinoblastoma cell areas of eyes from in-vivo study of retinoblastoma treatment. FIG. 3a shows cell areas from histological sections after H&E staining. FIG. 3b shows cell areas from histological section after staining with anti-human antibody.

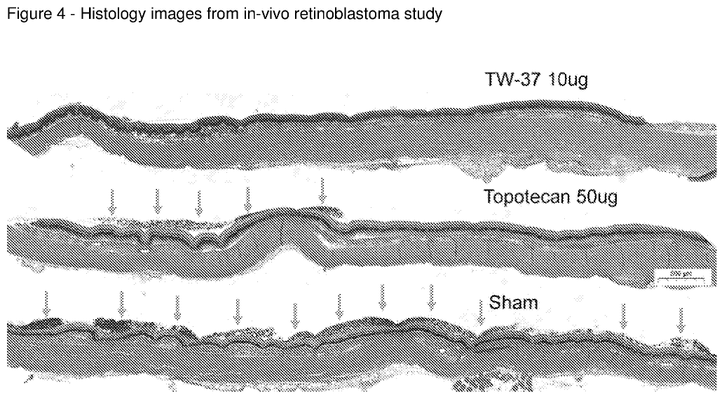

FIG. 4 shows histology images of the retinoblastoma cells on the retina from in-vivo study of retinoblastoma treatment. Arrows indicate evidence of retinoblastoma cell proliferation on the lining of retina.

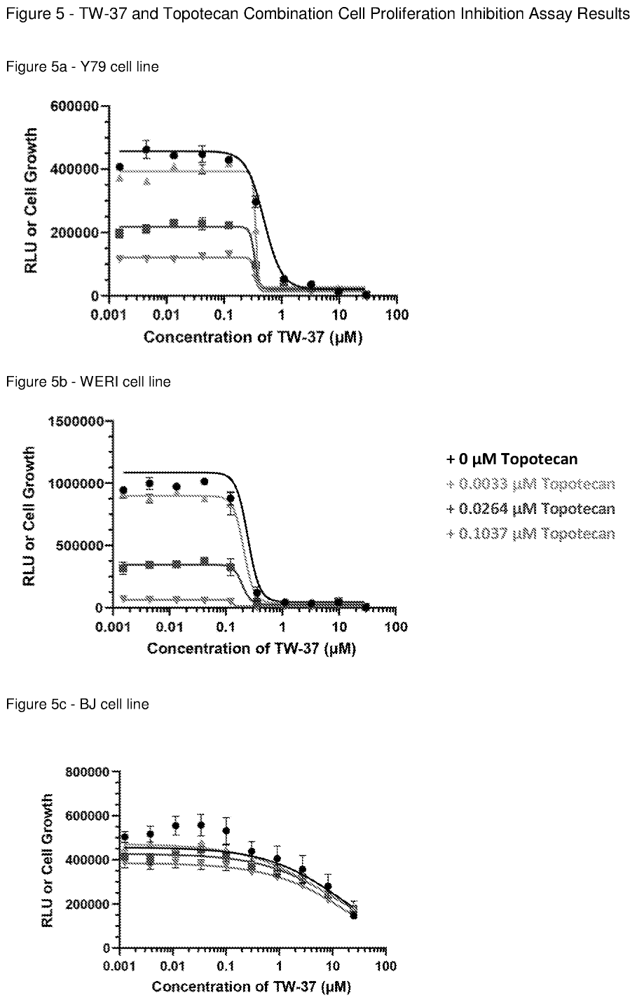

FIG. 5 shows the results of retinoblastoma cell proliferation inhibition assays with combination of a topoisomerase inhibitor (topotecan) and a Bcl-2 inhibitor (TW-37). FIG. 5a shows results for Y79 cell line. FIG. 5b shows results for WERI cell line. FIG. 5c shows results for BJ cell line.

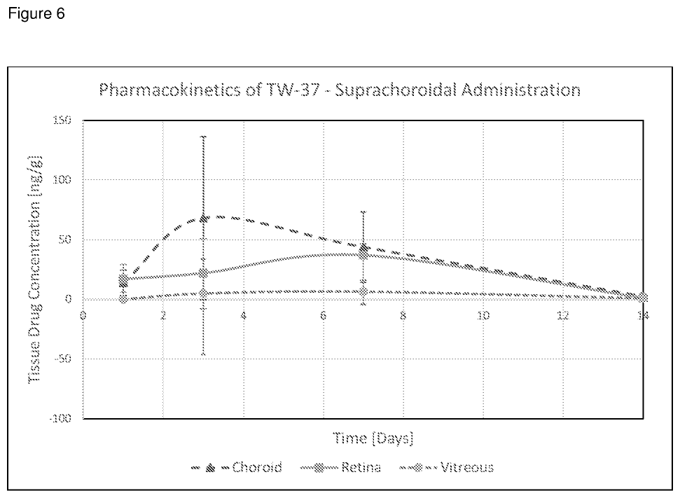

FIG. 6 shows the ocular pharmacokinetic results of suprachoroidal administration of a micelle formulation of TW-37 in rabbit eyes.

### EXAMPLE 1: RETINOBLASTOMA CELL PROLIFERATION INHIBITION ASSAYS WITH BCL-2 INHIBITOR AND TOPOISOMERASE Inhibitor

Testing of candidate compounds in a cell-based assay to determine the extent of cell inhibition with two human retinoblastoma cell lines (Y79 and WERI-Rb1) was performed. A normal fibroblast cell line (BJ) was used to identify active agents that selectively affect retinoblastoma. The cells were tested for mycoplasma contamination prior to use. Exponentially growing cells were plated in 384-well white, flat bottom, low flange, tissue culture treated assay plates and incubated overnight at 37° C. in a humidified 5% CO₂ incubator. DMSO inhibitor stock solutions were added the following day by manual pin transfer with 50SS pins to a top final concentration of 50 μM and 3 μM in 0.25% DMSO and then diluted 1/3 for a total of ten testing concentrations for each dilution scheme. Combined these two dilution schemes captured twenty data points from 50 μM to 0.2 nM. For Y79, the cells were plated to 1,000/cells per well in 25 microliters of complete media. For WERI-RB-1, the cells were plated to 2,000 cells/well in 25 microliters of complete media. For BJ, the cells were plated to 1,000/cells per well in 30 microliters of complete media. After addition of the candidate compounds, the number of cells was determined following a 72 hour incubation period using the Cell Titer Glo Reagent (Promega, Madison, Wis.). Luminescence was measured on a Clariostar plate reader (BMG Labtech). Assay endpoints were normalized from 0% (DMSO only) to 100% inhibition and fit to a semi-log plot using n=3 technical replicates and the four parameter variable slope algorithm in GraphPad Prism. The experiments were replicated a second time by a different operator to ensure reproducibility of the data.

Over 25 inhibitors of cellular pathways involved in cell growth and apoptosis were screened including Chetomin, Daprodustat, MK-8617, BAY-85-3934 (Molidustat), BAY-87-2243, 2-Methoxyestradiol, Vincristine-Sulfate, Calcitriol, Carboplatin, Melphalan, Etoposide, Lificiguat, Nutlin-3, Nutlin-3A, Idasanutlin, IOX2, RV1162, PTC-209, Cerdulatinib, Idarubicin, Cabatzitaxel, Romidepsin, TW-37, Flavopirodol, Obatoclax, BAY-61-3606, Topotecan, Doxorubicin. Evaluation of the cell inhibition and death curves provided an estimation of the active agent concentration for 50% cell inhibition (EC50). The results identified compounds with promising effectiveness against retinoblastoma with less toxicity to normal cells to provide a therapeutic range of treatment. The greatest effectiveness was found with Bcl-2 inhibitors (TW-37, sabutoclax), a topoisomerase inhibitor (topotecan) and a HDAC inhibitor (vorinostat).

Topotecan inhibits topoisomerase I activity by stabilising the topoisomerase I-DNA covalent complexes during S phase of cell cycle, thereby inhibiting re-ligation of topoisomerase I-mediated single-strand DNA breaks and producing potentially lethal double-strand DNA breaks when encountered by the DNA replication machinery. Topotecan demonstrated significant growth inhibition of retinoblastoma cells at low μM concentrations with very low toxicity to normal cells (p<0.001 at 1 μM). Topotecan demonstrated an EC50 of 0.069 μM for Y79 cell line, 0.039 μM for WERI cell line and >2.57 μM for BJ cell line. Two replicate assays were performed, confirming the results. (See FIGS. 1a, 1b, 1c).

TW-37 binds to the BH3 (Bcl-2 homology domain 3) binding groove of Bcl-2 and competes with pro-apoptotic proteins (such as Bid, Bim and Bad) preventing their heterodimerisation with Bcl-2, and therefore allowing these proteins to induce apoptosis. TW-37 demonstrated significant retinoblastoma cell kill at very low μM active agent concentration with very low toxicity to normal cells. (p<0.001 at 1 μM). TW-37 demonstrated an EC50 of 0.335 μM for Y79 cell line, 0.278 μM for WERI cell line and >8.76 μM for BJ cell line. Two replicate assays were performed, confirming the results (See FIG. 2a, 2b, 2c).

Vorinostat inhibits HDAC activity and inhibits class I and class II HDAC enzymes. The resulting accumulation of acetylated histones and acetylated proteins induces cell cycle arrest and apoptosis of some transformed cells. Vorinostat demonstrated an EC50 of 2.84 μM for Y79 cell line, 1.37 μM for WERI cell line and >54.4 μM for BJ cell line.

Sabutoclax is a pan-Bcl-2 family inhibitor that may activate caspase-3/7 and caspase 9, and may modulate Bax, Bim, PUMA and survivin expression. The agent provides reactivation of apoptosis mediated by several anti-apoptotic Bcl-2 family proteins. Sabutoclax demonstrated an EC50 of 0.316 μM for Y79 cell line, 0.211 μM for WERI cell line and >3.65 μM for BJ cell line.

### EXAMPLE 2: RETINOBLASTOMA IN VIVO MODEL TREATED WITH BCL-2 INHIBITOR AND TOPOISOMERASE INHIBITOR

Human retinoblastoma tumour cells, Y79 (ATCC@ HTB-18), were grown in media consisting of RPMI 1640 containing 20% FBS, L-glutamine 200 mM (100×), penicillin/streptomycin 5,000 U/ml and amphotericin B 250 μg/ml, to a target suspension density of 3×10⁵ cells/flask in 20 ml of media in a T75 flask. Thirty-two eyes in 16 immunosuppressed rabbits were inoculated with 200,000 cells in 30 μl of serum free media to the posterior retinal surface by intravitreal injection. The animals were studied in 4 groups of 8 eight eyes. Two groups were administered topotecan prepared in 30 μl of sterile saline injected intravitreally through a 29 gauge needle in the posterior region of the vitreous cavity near the tumour cells. One topotecan group was administered a 10 μg dose and a second group administered a 50 μg dose. The topotecan groups were administered the active agent formulation at 2, 3 and 4 weeks after tumour cell inoculation. One group was treated with 10 μg of TW-37 in 30 ul of DMSO injected in the vitreous cavity through a 29 gauge needle near the posterior retina adjacent to the tumour cells. The TW-37 group was administered the active agent formulation 3 and 4 weeks post tumour cell inoculation. A fourth group was treated with a sham injection of 30 μl sterile saline through a 29 gauge needle in the posterior region of the vitreous near the tumour cells at 2, 3 and 4 weeks after tumour cell inoculation. All animals were culled at 5 weeks to allow processing of the retina and retinoblastoma tumour cells on the retina for histological examination. Macro photography of the flat mounted retinae recorded tumour cell survival on the retinae and then representative sections were removed for fixation and subsequent processing for histology. All samples were cut at 5 μm and stained by H&E and replicate slides stained with human mitochondrial marker antibody to positively identify the human retinoblastoma cells. Slides were then scanned using a slide scanner (Histech or Hamamatsu S360) and retinoblastoma cell area on the retina quantified using CaseViewer software. The retinoblastoma cell areas from both H&E staining and antibody staining were lower compared to sham with both doses of topotecan treatment and the TW-37 treatment. The high dose topotecan treatment demonstrated statistically significant reduction in retinoblastoma cells in the eyes as compared to sham with p<0.01. The TW-37 treatment demonstrated statistically significant reduction in retinoblastoma cells in the eyes as compared to sham with p<0.01.

The cell area results are shown in FIGS. 3a and 3b. Representative histology images are shown in FIG. 4 with arrows indicating the retinoblastoma cells on the retina.

### EXAMPLE 3: RETINOBLASTOMA CELL PROLIFERATION INHIBITION ASSAYS WITH A COMBINATION OF BCL-2 INHIBITOR AND TOPOISOMERASE INHIBITOR

Using the cell assay method of Example 1, cell inhibition with combinations of TW-37 and topotecan was examined. Assays were performed with Topotecan titrated into the assay with TW-37 at constant concentration set to 0.662 μM. Topotecan concentrations of 0 μM, 0.0033 μM, 0.0264 μM and 0.1037 μM in DMSO were studied. The combination of TW-37 and Topotecan demonstrated additive inhibition of human retinoblastoma cell lines WERI and Y-79 with only slight toxicity to normal (BJ) cells. The results are shown in FIGS. 5a, 5b, 5c.

### EXAMPLE 4: MICELLULAR FORMULATION OF BCL-2 INHIBITOR WITH PEG-PHOSPHOLIPIDS

Micellular formulations of TW-37 were prepared. TW-37 solutions were prepared in DMSO at concentrations of 10 to 90 mM. PEG-phospholipid solutions were prepared with 1,2-distearoyl-sn-glycero-3-phosphoethanolamine-N-[methoxy(polyethylene glycol)-550] (18:0 PEG550 PE), 1,2-distearoyl-sn-glycero-3-phosphoethanolamine-N-[methoxy(polyethylene glycol)-1000] (18:0 PEG1000 PE), 1,2-dimyristoyl-sn-glycero-3-phosphoethanolamine-N-[methoxy(polyethylene glycol)-1000] (14:0 PEG1000 PE) dissolved in deionized water at concentrations of 5 to 45 mM. Equal volumes (20 μL) of MPEG solution and TW-37 solutions were combined in Eppendorf tubes and vortex mixed briefly at a molar stoichiometric ratio of TW-37 to PEG-phospholipid of 2:1. The solutions were examined for the presence of micelles by brightfield microscopy to identify the spherical micelles and changes over time such as loss of micelles, non-spherical particles, and aggregation. Formulations prepared with 18:0 PEG550 PE solutions at 5 and 15 mM (resulting in final formulation concentrations of 2.5 and 7.5 mM) with TW-37 solutions at 10 and 30 mM (resulting in final formulation concentrations of 5 and 15 mM) respectively demonstrated poor micelle formation. Formulations prepared with 18:0 PEG1000 PE solutions at 5, 15 and 45 mM (resulting in final formulation concentrations of 2.5, 7.5, and 22.5 mM) with TW-37 solutions at 10, 30, and 90 mM (resulting in final formulation concentrations of 5, 15, and 45 mM) respectively demonstrated micelle formation, with a greater number of micelles at the highest concentrations. However, the micelles showed limited stability with active agent escaping from the micelles to form crystals in the aqueous phase after 6 days at room temperature. Formulations prepared with 14:0 PEG1000 PE solutions at 5, 15 and 45 mM (resulting in final formulation concentrations of 2.5, 7.5, and 22.5 mM) with TW-37 solutions at 10, 30, and 90 mM (resulting in final formulation concentrations of 5, 15, and 45 mM) respectively demonstrated good micelle formulation with numerous micelles and no active agent crystals observed.

In similar study, equal volumes (20 μL) of MPEG solution in deionized water and TW-37 solutions in DMSO were combined in Eppendorf tubes and vortex mixed briefly at a molar stoichiometric ratio of TW-37 to PEG-phospholipid of 1:2. The solutions were examined for the presence of micelles by brightfield microscopy to identify the spherical micelles and changes over time such as loss of micelles, non-spherical particles, and aggregation. Formulations prepared with 18:0 PEG1000 PE solutions at 5, 15 and 45 mM (resulting in final formulation concentrations of 2.5, 7.5 and 22.5 mM) with TW-37 solutions at 2.5, 7.5, and 22.5 mM (resulting in final formulation concentrations 1.25, 3.75, 11.25 mM) respectively demonstrated micelle formulation, with a greater number of micelles at the highest concentrations. Formulations prepared with 14:0 PEG1000 PE solutions at 5, 15 and 45 mM (resulting in final formulation concentrations of 2.5, 7.5, 22.5 mM) with TW-37 at 2.5, 7.5, and 22.5 mM (resulting in final formulation concentrations of 1.25, 3.75, 11.25 mM) respectively demonstrated good micelle formulation with numerous micelles and no TW-37 crystals observed.

In a separate study, 18:0 PEG550 PE, 18:0 PEG1000 PE, 14:0 PEG1000 PE were prepared in deionized water at 10 and 15 mM. Solutions of TW-37 in DMSO were prepared at 3, 5, 7.5, and 10 mM. Equal 20 μL volumes of the solutions were mixed in an Eppendorf tube and vortex mixed to promote micelle formation. Brightfield microscopy showed formulations with 14:0 PEG1000 PE and TW-37 near 1:1 molar stoichiometry demonstrated the best micelle formation with greater number of micelles and no crystal formation indicating high association of the TW-37 with the micelles.

### EXAMPLE 5: MICELLULAR FORMULATION OF BCL-2 INHIBITOR WITH PEG-PHOSPHOLIPID 14:0 PEG1000 PE

Micellular formulations of TW-37 were prepared with PEG-phospholipid 14:0 PEG1000 PE as the micelle formulation excipient. TW-37 solutions were prepared in DMSO at concentrations of 7.5, 10, 15 and 20 mM. The PEG-phospholipid was prepared in deionized water at concentrations of 10, 15, 20, and 30 mM. Equal 20 μL volumes of the solutions were mixed in an Eppendorf tube and vortex mixed to promote micelle formation. The mixed formulations were examined for the presence of micelles by brightfield microscopy to identify the spherical micelles and changes over time such as loss of micelles, non-spherical particles, aggregation and active agent crystal formation in the aqueous phase, indicating escape of active agent from the micelles. The micellular formulations were stored in the dark at room temperature and examined by microscopy over a 3 week period. The following table characterized the formulations at 3 weeks.

The two most stable formulations were prepared with PEG-phospholipid solutions at 10 mM and TW-37 solutions at 7.5 mM (final formulation concentration of 5 mM and 3.75 mM from dilution) and PEG-phospholipid solutions at 30 mM and TW-37 at 20 mM (final formulation concentrations of 15 mM and 10 mM from dilution). In general, the formulations with greatest stability were observed with molar stoichiometries of approximately 1:1 PEG-phospholipid to TW-37, or slightly greater than 1:1 to provide some excess of PEG-phospholipid to TW-37.

| | PEG-phospholipid | | | | | | | |
|---|---|---|---|---|---|---|---|---|
| | TW-37 7.5 mM | | TW-37 15 mM | | TW-37 20 mM | | TW-37 30 mM | |
| [mM] | crystals | micelles | crystals | micelles | crystals | micelles | crystals | micelles |
| 10 | no | good | yes | good | yes | no | Yes | Full of |
| | | suspension | | amount of | | micelles | | crystals |
| | | | | micelles | | | | |
| 15 | yes | good | yes | good | yes | good | Yes | Full of |
| | | suspension | | suspension | | suspension, | | crystals |
| | | | | | | full of | | |
| | | | | | | micelles | | |
| 20 | no | few | no | good | no | full of | Yes | crystals |
| | | micelles | | suspension, | | micelles | | and |
| | | | | full of | | | | micelles |
| | | | | micelles | | | | |
| 30 | no | few | yes | few | no | less | No | full of |
| | | micelles | | micelles | | micelles | | micelles |
| | | | | | | than | | |
| | | | | | | normal | | |

### EXAMPLE 6: STABILITY OF MICELLULAR FORMULATIONS OF BCL-2 INHIBITOR

A formulation with equal volumes of PEG-phospholipid 14:0 PEG1000 PE at 30 mM and TW-37 at 15 mM was prepared to produce a final formulation of 15 mM PEG-phospholipid and 7.5 mM TW-37. The formulation was protected from light and stored at −80° C., −20° C., 4° C. and room temperature. The formulation demonstrated approximately full recovery at when stored at −80, −20 and 4° C. after 4 weeks, indicating formulation stability. The room temperature sample showed TW-37 content of 80.3% at four weeks.

### EXAMPLE 7: PHARMACOKINETIC STUDY OF BCL-2 INHIBITOR

A micellular formulation of TW-37 was prepared and administered into the suprachoroidal space of New Zealand White rabbits. A micellular formulation was prepared with an equal volumes of 4.8 mM TW-37 in DMSO added to 9.6 mM PEG-phospholipid 1,2-dimyristoyl-sn-glycero-3-phosphoethanolamine-N-[methoxy(polyethylene glycol)-1000] (14:0 PEG1000 PE) in deionized water for a final formulation of 2.9 mM TW-37 and 4.8 mM PEG-phospholipid. Both solutions were filter sterilized by passage through a sterile 0.2 micron nylon syringe filter into a sterile vial to produce a sterile formulation. The mixture was vortex mixed to produce a micelle suspension. A 25 μg dose of TW-37 in approximately 15 μL volume of the formulation was administered into the suprachoroidal space of twenty-four eyes in 12 rabbits. Eight eyes in 4 rabbits were administered 15 μL of a vehicle control into the suprachoroidal space, prepared identically to the active agent containing formulation but without TW-37. A flexible catheter with 250 micron OD and 140 micron ID was surgically introduced into the suprachoroidal space in the anterior region of the eye at the pars plana. The catheter was advanced posteriorly toward the posterior region of the suprachoroidal space. The catheter was configured to conduct light and provided illumination of the catheter tip and shaft to determine the catheter position and configuration by trans-scleral visualization. The illuminated tip of the catheter was used to position the catheter in the posterior region of the suprachoroidal space. The illuminated shaft of the catheter was used to manipulate and position the catheter to direct the injection toward the posterior region of the space. The study consisted of four groups, with each group consisting of six eyes administered the TW-37 formulation and two eyes administered the vehicle control. The eyes were examined by slit lamp in the anterior segment and by indirect ophthalmoscopy in the posterior segment prior to euthanasia for each time point at 1, 3, 7, 14 days post administration. The eyes were dissected and the vitreous, retina and choroidal separated and processed for TW-37 tissue concentration by LCMS. The tissue concentrations of TW-37 in the retina, choroid and vitreous are shown in FIG. 6.

The choroid demonstrated the highest level of TW-37 with the retina demonstrating a lower level of TW-37, generally following the pharmacokinetics of the choroid levels. The results indicate that the suprachoroidal space and choroid acted as a reservoir for the TW-37 and TW-37 passed into the retina to reach therapeutic levels. TW-37 had a peak concentration at 3 days in the choroid decreasing to near baseline at 14 days. TW-37 had a peak concentration in the retina at 7 days, decreasing to near baseline at 14 days. A single administration of TW-37 in the micellular formulation provided 14 days of tissue exposure of TW-37 to the target retina. The vitreous showed relatively low levels of TW-37 indicating low exposure to the anterior tissues of the eye and systemically.

1. A method for the treatment of retinoblastoma comprising administering a composition comprising a therapeutically active agent to a subject in need thereof by injection of the composition into the vitreous cavity, suprachoroidal space, supraciliary space or sub-Tenon's space of the eye adjacent to a retinoblastoma tumour.

2. The method of claim 1, wherein the therapeutically active agent is selected from the group consisting of a Bcl-2 inhibitor, a HDAC inhibitor or a topoisomerase inhibitor.

3. The method of claim 1 or claim 2, wherein the method comprises a further step of administering a composition comprising a therapeutically active agent, wherein the therapeutically active agent is selected from the group consisting of a Bcl-2 inhibitor or a topoisomerase inhibitor.

4. The method of any one of claims 1 to 3 wherein the Bcl-2 inhibitor is selected from the group consisting of TW-37, venetoclax, navitoclax, ABT-737, sabutoclax, obatoclax, ABT-263, oblimersen, AT101, SS5746, APG-1252, APG-2575, S55746 or UBX1967/1325.

5. The method of any one of claims 1 to 3 wherein the topoisomerase inhibitor is selected from the group consisting of topotecan, irinotecan, doxorubicin, irinotecan, daunorubicin, SN-38, voreloxin, belotecan or semisynthetic derivatives of podophyllotoxin (etoposide).

6. The method of any one of claims 1 to 3 wherein the HDAC inhibitor is selected from the group consisting of vorinostat, belinostat, panobinostat, romidepsin, entinostat, mocetinostat, CUDC-101, tacedinaline or nicotinamide.

7. The method of any one of claims 1 to 6, wherein the method comprises a further step of administering a composition comprising a DNA damaging agent.

8. The method of claim 7 wherein the DNA-damaging agent is selected from the group consisting of altretamine, bendamustine, busulfan, carboplatin, carmustine, chlorambucil, cisplatin, cyclophosphamide, dacarbazine, dactinomycin, ilfosfamide, lomustine, mechlorethamine, melphalan, oxaliplatin, procarbazine, streptozocin, temozolomide, thiotepa or trabectedin.

9. A composition comprising at least one therapeutically active agent selected from the group consisting of a Bcl-2 inhibitor, a HDAC inhibitor or a topoisomerase inhibitor for use in the treatment of retinoblastoma, wherein the composition is for administration into the vitreous cavity, suprachoroidal space, sub-Tenon's space, or supraciliary space adjacent to a retinoblastoma tumour in an eye.

10. The composition for use of claim 9, wherein the Bcl-2 inhibitor is selected from the group consisting of TW-37, venetoclax, navitoclax, ABT-737, sabutoclax, obatoclax, ABT-263, oblimersen, AT101, SS5746, APG-1252, APG-2575, S55746 or UBX1967/1325.

11. The composition for use of claim 9, wherein the topoisomerase inhibitor is selected from the group consisting of topotecan, irinotecan, doxorubicin, irinotecan, daunorubicin, SN-38, voreloxin, belotecan or semisynthetic derivatives of podophyllotoxin (etoposide).

12. The composition for use of claim 9, wherein the HDAC inhibitor is selected from the group consisting of vorinostat, belinostat, panobinostat, romidepsin, entinostat, mocetinostat, CUDC-101, tacedinaline or nicotinamide.

13. The composition for use of claim 9, wherein the composition comprises a Bcl-2 inhibitor, an excipient comprising an amphiphilic polymer, and an aqueous solution, wherein the Bcl-2 inhibitor is associated with the excipient in the form of micelles suspended in the aqueous solution.

14. The composition for use of claim 13, wherein the amphiphilic polymer comprises a polyethylene glycol conjugated lipid.

15. The composition for use of claim 14, wherein the polyethylene glycol conjugated lipid is selected from the group consisting of polyethylene glycol conjugated 1,2-dimyristoyl-sn-glycero-3-phosphoethanolamine (DMPE), conjugated 1,2-Dipalmitoyl-sn-glycero-3-phosphorylethanolamine (DPPE), conjugated 1,2-Distearoyl-sn-glycero-3-phosphorylethanolamine (DSPE) or conjugated 1,2-dioleoyl-sn-glycero-3-phosphoethanolamine (DOPE).

16. The composition for use of claim 15, wherein the polyethylene glycol in the polyethylene glycol conjugated lipid has a molecular weight range of 100 to 5000 Daltons.

17. The composition for use of any one of claims 13 to 16, where the Bcl-2 inhibitor is TW-37.

18. The composition for use of claim 17, wherein the concentration of the Bcl-2 inhibitor is in the range of 1.5 μM to 50 μM.

19. The composition for use of any one of claims 14 to 18, wherein the conjugated lipid in the polyethylene glycol conjugated lipid is 1,2-dimyristoyl-sn-glycero-3-phosphoethanolamine-N-[methoxy(polyethylene glycol)-1000].

20. The composition for use of claim 19 wherein the concentration of the conjugated lipid is in the range of 2.5 μM to 50 μM.

21. The composition for use of any one of claims 13 to 20, wherein the molar ratio of the Bcl-2 inhibitor to amphiphilic polymer is in the range of 1:2 to 2:1.

22. The composition for use of any one of claims 13 to 21, wherein the composition further comprises a topoisomerase inhibitor.

23. A kit comprising a Bcl-2 inhibitor, a HDAC inhibitor and/or a topoisomerase inhibitor for use in the treatment of retinoblastoma, wherein the Bcl-2 inhibitor and the topoisomerase inhibitor are for separate, simultaneous or sequential administration.

24. The kit of claim 23, wherein the Bcl-2 inhibitor is selected from the group consisting of TW-37, venetoclax, navitoclax, ABT-737, sabutoclax, obatoclax, ABT-263, oblimersen, AT101, SS5746, APG-1252, APG-2575, S55746 or UBX1967/1325.

25. The kit of claim 23, wherein the topoisomerase inhibitor is selected from the group consisting of topotecan, irinotecan, doxorubicin, irinotecan, daunorubicin, SN-38, voreloxin, belotecan or semisynthetic derivatives of podophyllotoxin (etoposide).

26. The kit of claim 23, wherein the HDAC inhibitor is selected from the group consisting of vorinostat, belinostat, panobinostat, romidepsin, entinostat, mocetinostat, CUDC-101, tacedinaline or nicotinamide.

27. The use of a Bcl-2 inhibitor, a HDAC inhibitor or a topoisomerase inhibitor in the manufacture of a medicament for the treatment of retinoblastoma by administration into the vitreous cavity, suprachoroidal space or sub-Tenon's space adjacent to a retinoblastoma tumour in an eye.

28. The use of claim 27, wherein the Bcl-2 inhibitor is selected from the group consisting of TW-37, venetoclax, navitoclax, ABT-737, sabutoclax, obatoclax, ABT-263, oblimersen, AT101, SS5746, APG-1252, APG-2575, S55746 or UBX1967/1325.

29. The use of claim 27, wherein the topoisomerase inhibitor is selected from the group consisting of topotecan, irinotecan, doxorubicin, irinotecan, daunorubicin, SN-38, voreloxin, belotecan or semisynthetic derivatives of podophyllotoxin (etoposide).

30. The use of claim 27, wherein the HDAC inhibitor is selected from the group consisting of vorinostat, belinostat, panobinostat, romidepsin, entinostat, mocetinostat, CUDC-101, tacedinaline or nicotinamide.

31. A kit comprising a composition comprising at least one therapeutically active agent and a cannulation or catheterization device for use in the treatment of retinoblastoma in an eye, wherein the at least one therapeutically active agent is selected from the group consisting of a Bcl-2 inhibitor, a HDAC inhibitor or a topoisomerase inhibitor and wherein the cannulation or catheterization device is configured for delivery of the composition to the suprachoroidal space or supraciliary space.

32. The kit of claim 31, further comprising a pharmaceutically acceptable diluent.

33. The kit of claim 31, wherein the cannulation or catheterization device is configured to deliver injection volume in a range from 10 to 100 microliters.

34. A method for preparing the composition for use of claims 13 to 22 comprising:
mixing the Bcl-2 inhibitor with an organic solvent to dissolve the Bcl-2 inhibitor;
filtering sterilely the mixture;
adding the organic solvent mixture to a volume of sterile filtered aqueous solution containing the amphiphilic polymer excipient; and
mixing the formulated composition to produce Bcl-2 inhibitor containing micelles in an aqueous solution.

35. The method of claim 34, wherein the Bcl-2 inhibitor is TW-37.

36. The method of claim 34 or claim 35, wherein the amphiphilic polymer excipient is 1,2-dimyristoyl-sn-glycero-3-phosphoethanolamine-N-[methoxy(polyethylene glycol)-1000].

37. The method of claims 34 to 36, wherein the organic solvent is DMSO.
